(19) United States
(12) Patent Application Publication
(10) Pub. No.: US 2014/0024883 A1
(43) Pub. Date: Jan. 23, 2014

(54) DEVICE AND METHOD FOR STIMULATING EYE MOVEMENT

(71) Applicant: Helene Rosenzweig, West Hartford, CT (US)

(72) Inventor: Helene Rosenzweig, West Hartford, CT (US)

(73) Assignee: UNIVERSITY OF SOUTH FLORIDA, Tampa, FL (US)

(21) Appl. No.: 14/041,090

(22) Filed: Sep. 30, 2013

Related U.S. Application Data
(63) Continuation of application No. PCT/US2012/031838, filed on Apr. 2, 2012.
(60) Provisional application No. 61/469,968, filed on Mar. 31, 2011.

(51) Int. Cl. A61M 21/00

(52) U.S. Cl. CPC A61M 21/00
USPC 600/27

(57) ABSTRACT

A device for stimulating eye movement in a subject is presented. Stimulating eye movement through Accelerated Resolution Therapy (ART) allows the processing of information and elimination of negative body sensations. In an embodiment, a mechanical device is presented in which a physical object is attached to a motion mechanism which moves horizontally from left to right. In another embodiment, an electrical device is presented in which a computer-simulated object travels horizontally across a visual component. In a further embodiment, a computer software application is presented to track eye movements. The subject tracks the movement of the object with his eyes for a specific number of cycles in order to eliminate negative body sensations such as post-traumatic stress disorder. The device of the present invention may be used for sensation processing by the general public and may be used for ART by a trained therapist.

### CROSS REFERENCE TO RELATED APPLICATIONS

This application is a continuation of prior filed International Application, Serial Number PCT/US2012/031838 filed on Apr. 2, 2012, which claims priority to U.S. Provisional Application No. 61/469,968 entitled “Device and Method of Stimulating Eye Movement”, filed Mar. 31, 2011, the contents of which are hereby incorporated by reference into this disclosure.

### FIELD OF INVENTION

This invention relates to devices used to treat psychological trauma and mental disorders. Specifically, the invention provides a device and method for stimulating eye movements in a subject in order to process information and eliminate negative body sensations.

### BACKGROUND OF THE INVENTION

Symptoms of psychological trauma are prevalent, chronic, and highly disabling among adult populations in North America. Various types of incidents such as wars, natural disasters, traffic accidents, rapes, violence, sexual abuse, physical abuse, etc. can have a serious effect on a person's mental health. These individuals may have painful memories causing post-traumatic stress disorder (PTSD), phobias, panic attacks, emotional disorders, etc. in psychological trauma often a trigger, such as a traumatic or emotional occurrence, is either unprocessed or improperly processed by the human brain and can result in behavioral changes. As a person develops, the images related to the trauma may become buried deeply within the person's mind and may be subsequently triggered by future events. Existing treatment regimens have included the use of psychotherapeutic drugs which have variable success and often require a long duration of therapy.

Posttraumatic Stress Disorder (PTSD) is a prevalent, disabling anxiety disorder that may occur after witnessing a traumatic event which then evokes a combination of re-experiencing, avoidance, numbing, and/or arousal symptoms. (American Psychiatric Association. (2000). Diagnostic and statistical manual of mental disorders Revised 4th ed. Washington, D.C.). Among adults 18 and older in the U.S. population, lifetime and past year prevalence of PTSD have been estimated at 6.8% and 3.5%, respectively. (Kessler, R. C., Berglund, P., Delmer, O., Jin, R., Merikangas, K. R., & Walters, E. E. (2005). Lifetime prevalence and age-of-onset distributions of DSM-IV disorders in the National Comorbidity Survey Replication. Archives of General Psychiatry, 62, 593-602). The prevalence of PTSD varies considerably among subgroups, and is much higher than the national average among women (National Comorbidity Survey 2005, NCS-R appendix tables: Table 1. Lifetime prevalence of DSM-IV/WMH-CIDI disorders by sex and cohort. Table 2. Twelve-month prevalence of DSM-IV/WMH-CIDI disorders by sex and cohort), U.S. service members (Tanielian, T., & Jaycox, L. (2008). Invisible Wounds of War: Psychological and Cognitive Injuries, Their Consequences, and Services to Assist Recovery. Santa Monica, Calif.: RAND Corporation), and veterans (Kang, H. K., Natelson, B. H., Mahan, C. M., Lee, K. Y., & Murphy, F. M. (2003). Post-traumatic stress disorder and chronic fatigue syndrome-like illness among Gulf War veterans: A population-based survey of 30,000 veterans. American Journal of Epidemiology, 157, 141-148; Kulka, R. A., Schlenger, W. A., Fairbanks, J. A., Hough, R. L., Jordan, B. K., Marmar, C. R., & Cranston, A. S. (1990)). Trauma and the Vietnam War generation: Report of findings from the National Vietnam Veterans Readjustment Study. New York). Comorbidity rates of PTSD with other medical, mental health and substance abuse disorders may exceed 80%. (Kessler, R. C., Sonnega, A., Bromet, E., Hughes, M., & Nelson, C. B. (1995). Posttraumatic stress disorder in the national comorbidity survey. Archives of General Psychiatry, 52, 1048-1060). The most common disorders seen in PTSD patients are depressive disorders, panic disorder, other anxiety disorders, substance abuse or dependence, and personality disorders. (Bisson, J. I. (2007). Post-traumatic stress disorder. BMJ, 334, 789-793; Davidson, J. R. T., & Foa, E. B., (Eds.). (1993). Posttraumatic Stress Disorder: DSM-IV and beyond. Washington, D.C.: American Psychiatric Press; Kulka, R. A., Schlenger, W. A., Fairbanks, J. A., Hough, R. L., Jordan, B. K., Marmar, C. R., & Cranston, A. S. (1990)).

In addition, PTSD is associated with indicators of poor general health—increased physical symptoms, high somatic symptom severity, and higher numbers of sick call visits and missed workdays. (Hoge, C. W., Terhakopian, A., Castro, C. A., Messer, S. C., & Engel, C. C. (2007). Association of posttraumatic stress disorder with somatic symptoms, health care visits, and absenteeism among Iraq war veterans. American Journal of Psychiatry, 164, 150-153). This high prevalence of psychological and physical comorbidities is associated, in turn, with impaired family relationships, work relationships, and friendships, which may contribute to the reported increased risk of suicidal behavior. (VA National Center for PTSD, Relationships and PTSD; Oquendo, M. A., Friend, J. M., Halberstam, B., Brodsky, B. S., Burke, A. K., Grunebaum, M. F., Mann, J. J. (2003). Association of comorbid posttraumatic stress disorder and major depression with greater risk for suicidal behavior. American Journal of Psychiatry, 160, 580-582).

First line evidence-based cognitive-behavioral treatments (CBT) for PTSD include Cognitive Processing Therapy (CPT), Prolonged Exposure (PE), and Eye Movement Desensitization and Reprocessing (EMDR) which lead to clinically improved outcomes in approximately 50% of all treated cases. (Foa, E. B., Keane, T. M., & Friedman, M. J. (2009). Effective treatments for PTSD: Practice guidelines from the International Society for Traumatic Stress Studies. New York: Guilford; Institute of Medicine. (2008). Treatment of posttraumatic stress disorder: An assessment of the evidence. Washington, D.C.; Ursano, R. J., Bell, C., Eth, S., Friedman, M. J., Norwood, A. E., & Pfefferbaum, B. (2004). Practice guideline for the treatment of patients with acute stress disorder and posttraumatic stress disorder. American Journal of Psychiatry (Suppl), 161).

CPT focuses on challenging and modifying maladaptive beliefs related to the trauma, while including a written exposure component. (Resick, P. A., & Schnicke, M. K. (1996). Cognitive processing therapy for rape victims: A treatment manual. Newbury Park, Calif.: Sage Publications).

PE includes imaginal and in vivo exposure to safe situations that have been avoided because they elicit traumatic memories. (Foa, E. B., & Rothbaum, B. O. (1998). Treating the trauma of rape: Cognitive behavioral therapy for PTSD. New York: Guilford). Prolonged exposure therapy takes a minimum of twelve sessions and the client must be exposed to the problem images repeatedly.

EMDR engages in imaginal exposure to a trauma while simultaneously performing saccadic eye movements. (Chemtob, C. M., Tolin, D. F., van der Kolk, B., & Pitman, R. K. (2000). Eye movement desensitization and reprocessing. New York: Guilford; Shapiro, F. (2001). Eye movement desensitization and reprocessing: Basic principles, protocols and procedures (2nd ed.). New York: Guilford).

In addition to CBT, treatment with selective serotonin reuptake inhibitors (SSRI's) has the strongest evidence for treating PTSD and is specifically approved by the FDA for this purpose. (Brady, K., Pearlstein, T., Asnis, G. M., Baker, D., Rothbaum, B., Sikes, C. R., & Farfel, G. M. (2000). Efficacy and safety of sertraline treatment of posttraumatic stress disorder: a randomized controlled trial. JAMA, 283, 1837-1844). However, SSRI's usually do not eliminate all symptoms, and potential side effects need to be continuously monitored during use. (VA National Center for PTSD. (2012). Clinicians Guide to Medications for PTSD). Despite the established efficacy of pharmacotherapy and cognitive behavioral therapies, there remains intense interest in finding new ways to advance PTSD treatment effectiveness, accessibility, and delivery. This includes drawing on strengths of individual therapies, and ideally, reducing overall treatment length. These conditions led to the development of a new exposure-based therapy in 2008 known as Accelerated Resolution Therapy (ART) that uses eye movements and is the focus of this report. This therapy was developed to be highly procedural and to be administered to patients in a short period of time (1-5 sessions in 3 weeks).

As briefly stated above, prior art in the area of stimulating eye movement in a subject to treat various negative sensations in the body includes Rapid Eye Technology in which a technician simulates rapid eye movement (REM) with an eye-directing device moving in a neuro-linguistic pattern in front of a subject's eyes. Various mind areas are stimulated by a special eye movement and blinking pattern that is coupled with rapid verbal input that releases stress from the subject's body. This technique is not automated and must be done by a technician trained in the method using a physical object (a wand) to guide the subject's eyes. Further, this technique uses both eye movements and blinking that is coupled with verbal input.

Eye Movement Desensitization and Reprocessing (EMDR) is a prior art method which typically involves sweeping eye motion in a lateral movement while processing traumatic memories of earlier events in a person's life. In EMDR both eyes of a subject track a continuously moving hand in a horizontal plane from left to right alternatively the therapist may use clickers in the hand.

The therapy developed by the inventor, Accelerated Resolution Therapy (ART), for use with the device disclosed herein, differs in several ways from EMDR therapy. EMDR is more of a free association method in which clients are asked to note their internal experiences with minimal clinical intervention. At the end of every set of eye movements clients are asked about their internal experience which automatically brings new pieces of information to mind (free association). There is no set frequency for using the eye movements and the eye movement process is intentionally kept varied. Sensations may be processed after every three or four sets of eye movements. Clinicians are not supposed to explore the meaning of any symbols, memories, thoughts, feelings, etc. that are expressed by the client during the session. If a client “gets stuck” while processing a problem, the clinician can only offer minimal suggestion. Clients spend very little time exposed to the details of the traumatic event. EMDR is not a regimented, procedural approach thus ensuring that no two treatment sessions will be the same. EMDR also may take several sessions over a long period of time to achieve results.

In ART, in contrast to EMDR, the clinician guides clients and is directive with many interventions specific to ART that facilitate and enhance processing in every step of the process. ART is a procedural approach that focuses sequentially on the traumatic images from the presenting problem. The clinician instructs the client on how to eliminate the distressing images or connect the images to positive images of the client's choice using a technique called Voluntary Memory/Image Replacement (VMR/VIR). VMR/VIR is an intervention which helps the client eliminate negative mental images from view which in turn eliminates negative sensations and triggers that are the cause of the client's problem. As the disturbing images disappear, the symptoms of the problem often disappear with them. The narrative form of the memory stays in a different location in the brain so that the story is remembered in a narrative form while the images are either very dim or not able to be accessed. It is important to the ART therapist that they try and complete the scene(s) that are at hand and involve the presenting problem before exploring other material exhumed from a free association method. Processing the presenting scene often takes care of many other scenes that were associated with it. It usually takes only one to three scenes to completely process one client problem or issue. If an underlying problem is suspected, the therapy uses an intervention called a “Scene Match” to go directly to the underlying problem by having the client associate their sensations with an earlier scene in their life. The earlier scene is often processed along with the presenting problem scene.

ART also uses specific numbers of eye movements in each set to process through a problem. Sensations are processed after the completion of each set of eye movements to ascertain whether additional eye movements are needed or if the problem has been fully processed.

One issue with both EMDR and ART administration for the therapist is maintaining a controlled rate of speed and an accurate path for their hand/stylus for prolonged periods of time which may result in inconsistent eye movements. Certain devices have been created to alleviate this problem. However, these devices mainly consist of a plurality of light emitting diodes (LEDs) which light up sequentially to allow a subject to follow the illuminated LEDs. The subject does not have a choice of different objects to follow. Having a choice of objects to follow is beneficial to the subject for a variety of reasons. For example, in the case of a child suffering from trauma, it may be easier for the child to identify with and feel more comfortable with following an object such as a teddy bear as opposed to following a light. It is also easier for a subject to keep focus when following an actual object as opposed to following only a light.

One prior art device used in conjunction with EMDR therapy is U.S. Pat. No. 5,343,261 which discloses a device for inducing eye movement in a subject by using a horizontal line of sequentially illuminating light emitting diodes (LED). These LEDs are mounted on a horizontal surface that is placed at the eye level of the subject. The LEDs are controlled manually by a control knob and the activation of each individual LED is tracked by a counter. This device is not portable; does not allow for different patterns to be tracked; and does not allow the subject to choose and modify the object to be tracked.

Another prior art device, U.S. Pat. No. 6,056,403, discloses a device having a hand held screen that is capable of displaying a multiplicity of LEDs that can be illuminated sequentially to stimulate eye movement. Different visual patterns, besides a horizontal line, may be executed with this device. While this device is portable, it does not allow the subject to choose and modify the object being tracked. In addition, this device does not offer voice instruction or white noise.

While the above prior art devices may be used in some eye movement therapies, they have limitations in not permitting the client to choose the object to be tracked, not being portable, not tracking eye movements, and not offering voice instruction. What is needed therefore is a portable device that can be used in conjunction with ART in which the object to be tracked can be chosen by the client and the eye movements tracked.

### SUMMARY OF INVENTION

The present invention provides a device for stimulating eye movement that may be used in conjunction with ART. A device for stimulating eye movement in a subject is presented comprising: a housing having a first end and a second end; a motion mechanism disposed within the housing; and an object permanently or removably attached to the motion mechanism; wherein activation of the motion mechanism moves the object horizontally back and forth between the first and second ends of the housing; whereby a subject follows the horizontal movement of the object for a predetermined number of cycles to stimulate eye movement and process negative sensations.

The motion mechanism may be a pulley system comprised of a first pulley disposed within the first end of the housing; a second pulley disposed within the second end of the housing wherein the second pulley is rotatably attached to the motor; a circular belt extending between the first pulley and the second pulley; wherein activation of the motor rotates the second pulley to move the belt in one direction and wherein movement of the belt rotates the first pulley in the same direction as the rotation of the second pulley.

The device may have at least one stand positioned beneath the housing wherein the object is positioned at eye level with the subject.

The device may also have a slot disposed along the first side of the housing wherein the slot extends from substantially the first end of the housing to substantially the second end of the housing.

The device may also have a motor attached to the motion mechanism; a controller to provide voltage to the motor; a power source electrically connected to the controller; and a microprocessor electrically interfaced with the controller.

In another embodiment, a device for stimulating eye movement in a subject is presented comprising: a control component having a top side and a bottom side; a microprocessor contained within the control component; a visual component positioned on the top side of the control component; a power source electrically attached to the control component; and a plurality of switches on the top side of the control component wherein the plurality of switches send a signal to the microprocessor to control selecting and modulating the appearance and speed of an object projected on the visual component and tracking movement of the object on the visual component; wherein the object moves in a substantially horizontal line across the visual component; whereby a subject follows the horizontal movement of the object for a predetermined number of cycles to stimulate eye movement and process negative sensations.

The device may also have a plurality of speakers disposed within the control component and a plurality of switches positioned on the top side of the control component for controlling the volume and type of sound emanating from the speakers. The power source may be a battery.

In a further embodiment, a computer-implemented therapeutic software application is presented, the software accessible from a non-transitory media and providing instructions for a computer processor to generate a moving graphic indicia on a computer display, the instructions comprising: displaying the graphic indicia on the computer display; moving the graphic indicia on a horizontal plane in a first direction; responsive to the graphic indicia meeting a first predetermined vertical boundary of the computer display, reversing the direction the graphic indicia to move in the opposing direction until it meets a second predetermined vertical boundary wherein it again reverses direction, the back and forth movement comprising a cycle; and repeating the cycle a predetermined number of times wherein an end user observing the cycling graphic indicia receives therapeutic benefit.

In another embodiment, a method of stimulating eye movement in a subject is presented comprising: providing a device with an object visually displayed at eye level of the subject wherein the device moves the object horizontally in a first direction to a first predetermined stop point wherein at the first stop point the device moves the object in a second opposite direction to a second predetermined stop point with this back and forth movement comprising a cycle; instructing the subject to follow the object with only their eyes; repeating the cycle for a predetermined number of times wherein the number of times is between about 20 and about 40; and assessing resolution of a problem; wherein if the problem is not resolved, the cycle is repeated. The object may be moving at a predetermined speed. The object may be a physical object or a computer-generated object.

In use, the device enables an object of the subject's choosing to travel horizontally back and forth across the device for a predetermined number of cycles. The subject follows the object with his/her eyes while concentrating on distressing images to process out negative sensations.

### BRIEF DESCRIPTION OF THE DRAWINGS

For a fuller understanding of the invention, reference should be made to the following detailed description, taken in connection with the accompanying drawings, in which:

FIG. 1 is an image depicting a front view of one embodiment of the mechanical device.

FIG. 2 is an image depicting a back view of one embodiment of the mechanical device.

FIG. 3 is an image depicting a top view of one embodiment of the mechanical device.

FIG. 4 is an image of one embodiment of the electronic device showing the control component and the visual display component of the device.

FIG. 5 is an image depicting the background screen.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments by which the invention may be practiced. It is to be understood that other embodiments may be utilized and structural changes may be made without departing from the scope of the invention.

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose.

The terms “subject” and “client” are used interchangeably herein to refer to a human who is undergoing ART therapy and/or using an embodiment of the device disclosed herein.

The term “motion mechanism” as used herein refers to any mechanism which is capable of moving an object attached thereto in a back and forth horizontal direction. One of ordinary skill in the art would be able to envision various systems which could be used in conjunction with the mechanical device of the present invention. Examples of a motion mechanism include, but are not limited to, a pulley system, a wheel with teeth moveably attached to an elongated horizontal member, and an elongated screw system with a rotatably attached rod.

The term “stop mechanism” as used herein refers to any mechanism which is capable of stopping forward or backward movement of the object to be tracked by the subject at a certain position/point and reversing the direction of the movement of the object to the opposite direction. One of ordinary skill in the art would be able to envision various systems which could be used in conjunction with the mechanical device of the present invention. Examples of the stop mechanism include, but are not limited to, a pressure sensor, a laser system, and a timing system. If a pressure sensor is used, once the component to which the object is attached makes contact with the pressure sensor, the direction the object is traveling will reverse. In the case of a laser system, a laser beam is directed across the interior of the housing of the device. Once the component to which the object is attached passes through the laser beam (so that the laser beam is broken), the direction the object is traveling will reverse. If a timing mechanism is used, certain preset times may be programmed into the timing mechanism so that once the predetermined amount of time has elapsed, the direction the object is traveling will reverse.

The inventor has previously developed a therapy, Accelerated Resolution Therapy (ART), which uses eye movements to process information. In addition to the processing of information, negative body sensations can often be eliminated by the use of the proper amount of these eye movements. The eye movements are thought to be conducive to sorting through problems quickly by increasing the integration of activities in the left and right sides of the brain. The eye movements assist the client in processing information by inducing a deep feeling of relaxation and by making new connections to information stored in the brain. ART uses specific and unique interventions that involve a specific number of eye movements to facilitate processing of information. These specific number of eye movements, while enhancing the processing of information, also prevent flooding of too much information at one time.

ART is interactive as a dialogue takes place between the ART therapist and the subject. ART with a trained ART therapist is generally prescribed for those subjects who have a specific problem to work through as opposed to negative sensations only. Those subjects with a specific problem to work through benefit from the reciprocity received from resolving a problem “scene” with the direction of the ART therapist.

Currently, therapists who are trained in the ART technique use their hand to guide the client in using eye movements. The device of the present invention will allow the therapist to use the device to guide the eye movements as opposed to their hand thus alleviating strain on the therapist as well as providing a constant rate and speed for the eye movement sets. The use of the device for processing out a problem for a client is as effective, if not more effective, than the use of ART therapy using the therapist's hand.

An ART therapist may teach a subject how to use eye movements to alleviate negative sensations in the body or to assist subjects having sleep issues. The device of the present invention can be used by both the ART therapist in an ART session as well as by a subject on their own. When the eye movements are used for sensations only, it is termed “SAF-T” which stands for Sensation Awareness Focused Training.

The device, when used in conjunction with ART therapy, is designed to process out client problems and, as part of that process, will assist with physical sensations that cause discomfort. For example, the device can be used for physical sensations associated with anxiety, phobias, stress-induced headaches, heartburn, post-traumatic stress disorder, depression, sexual abuse trauma, phobias, obsessive compulsive disorder, grief, addiction cravings, and other mental health problems such as Attention Deficit Disorder. The device, which makes new connections in the brain, may also be used for cognitive enhancement including learning and memory improvement for both impaired and unimpaired individuals, including those suffering from dementia. Students may also benefit from using the device for memory enhancement and learning.

Device 10 is generally comprised of housing 30 having first end 31 and second end 32; motion mechanism 115 disposed within housing 30; and object 100 permanently or removably attached to motion mechanism 115. Housing 30 may be constructed of any synthetic or natural material known to those of ordinary skill in the art including, but not limited to, metal, plastic, and wood. Motion mechanism 115 may alternative be positioned to be attached to the front of housing 30 as opposed to being positioned within housing 30. Many embodiments are possible for configurations of motion mechanism 115 and housing 30. These embodiments are meant to be encompassed in the disclosure of the invention provided herein. Below are examples of specific embodiments of the invention which are intended to be merely exemplary and are not meant to be construed in a limiting sense.

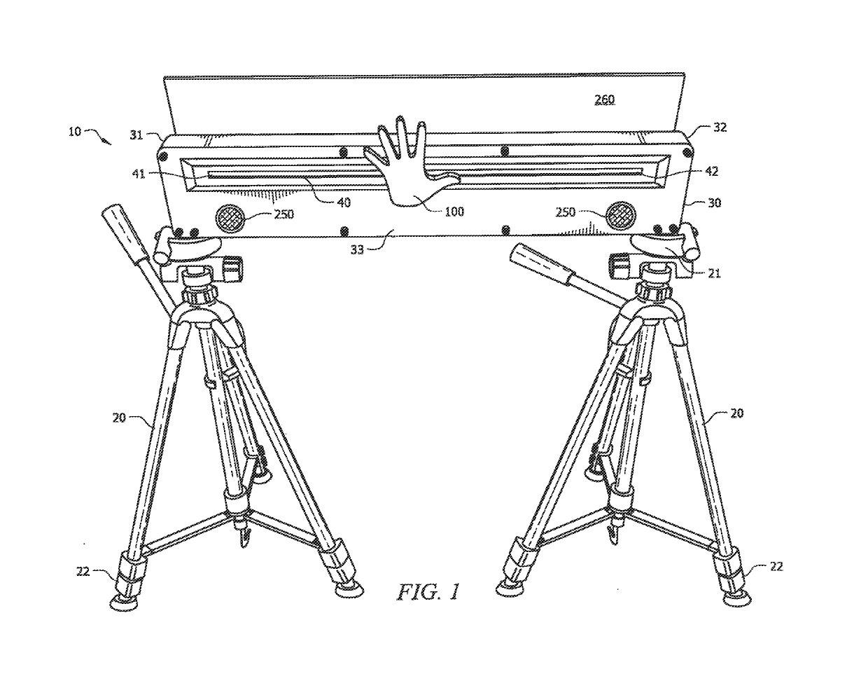

In one embodiment represented by FIGS. 1-3, mechanical device 10 for use in stimulating eye movement during ART is presented. Stand 20 has top surface 21 and bottom 22. Housing 30 is positioned on top surface 21 of stand 20. One stand 20 may be positioned beneath the center of housing 30 or alternatively, two stands 20 may be used in which one stand 20 is positioned under first end 31 of housing 30 and the other stand 20 is positioned under second end 32 of housing 30. Alternatively, housing 30 may be removably mounted on a surface such as a wall or table as long as object 100 is positioned at eye level with the subject.

As depicted in FIG. 1, first side 33 of housing 30 contains slot 40 which extends horizontally from first end 31 of housing 30 to second end 32 of housing 30. First end 41 of slot 40 is positioned at substantially first end 31 of housing 30 and second end 42 of slot 40 is positioned at substantially second end 32 of housing.

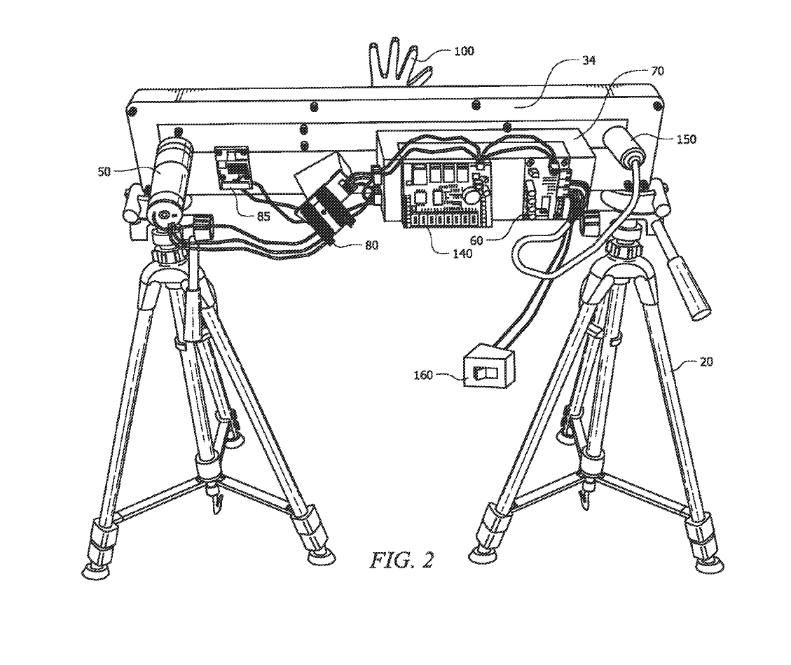

As depicted in FIG. 2, back side 34 of housing 30 contains motor 50. Motor 50 may be any type of motor known by those of ordinary skill in the art to be capable of powering device 10. For example, motor 50 can be a 12 volt gear motor. An example of a motor that may be used in the present invention is Phidget 3252.

Motor 50 is electrically connected to controller 60. Controller 60 may be a circuit board that is electrically connected to power source 70. Controller 60 is capable of controlling the direction, velocity and acceleration of motor 50. Preferably, controller 60 has multiple digital, analog and encoder inputs. An example of a controller that may be used in the present invention is Phidget 1065.

In an embodiment, motor 50 may be connected to heat sink 80 which is then connected to controller 60. In this embodiment, amplifier 85 may be connected to motor 50 and heat sink 80. Heat sink 80 may be used as a voltage source to generate the power to turn motor 50. Amplifier 85 may be used to amplify the small command voltage from controller 60 to drive motor 50.

Power source 70 provides electrical power to device 10 including providing power to controller 60, motor 50, and microprocessor 140. Power source 70 may receive electrical current through a standard wall outlet or alternatively through a battery. Power source 70 may be an AC converter that is capable of converting AC to DC for powering motor 50. Once of ordinary skill in the art would readily identify power sources that can be used with device 10.

Microprocessor 140 is electrically interfaced with controller 60. This electrical interface may be through a USB cable or other means known to those of ordinary skill in the art. Microprocessor 140 may be any microprocessor capable of being hardwired or running software to run a general control code in which the position of object 100 is measured and if object 100 is at the edge then the direction of motor 50 voltage is switched; otherwise motor 50 is kept moving in the same direction. If the position of object 100 switches at first end 31 of housing 30 then increment counter for the number of eye movements. Microprocessor 140 may be a stand-alone computer or alternatively, microprocessor 140 may be a computer that is onboard device 10.

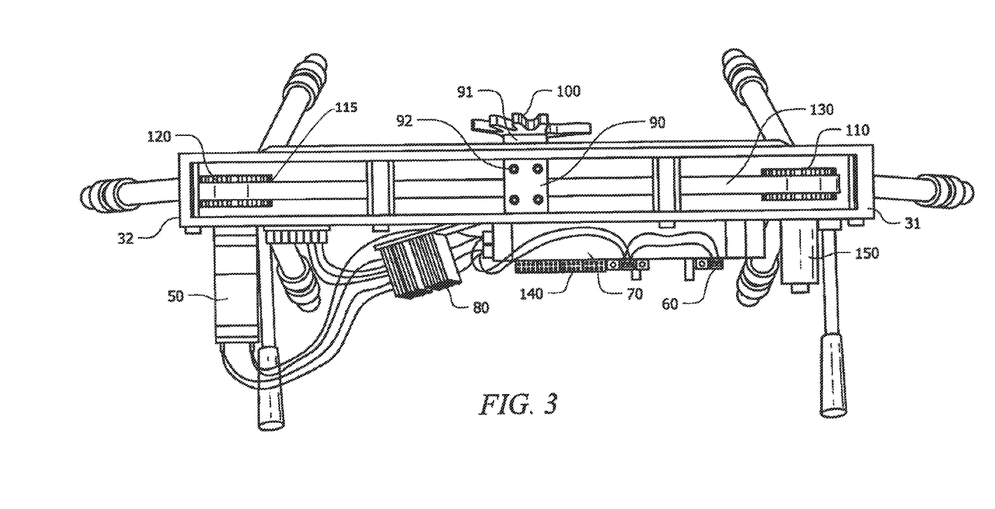

As depicted in FIG. 3, motion mechanism 115 may be positioned within housing 30. This motion mechanism may take many forms. Those of ordinary skill in the art would be capable of identifying different forms of motion mechanisms capable of being used with device 10. In an embodiment, motion mechanism is comprised of a pulley system comprised generally of first pulley 110, second pulley 120 and circular belt 130. First pulley 110 may be disposed at first end 31 of housing 30 and rotatably attached to encoder 150 while second pulley may be disposed at second end 32 of housing 30 and rotatably attached to motor 50. One end of circular belt 130 may partially wrap around first pulley 110 while opposite end of circular belt 130 may partially wrap around second pulley 120. Circular belt 130 is taut as it extends between first pulley 110 and second pulley 120. When motor 50 is activated to operate in a first direction, second pulley 120 turns in a first direction thereby moving circular belt 130 in that same direction. The movement of circular belt 130 in turn rotates first pulley 110 in the same direction as second pulley 120. Conversely, when motor 50 is sent the signal to operate in a second direction, second pulley 120 turns in the opposite direction thereby moving circular belt 130 in that same opposite direction. The movement of circular belt 130 in turn rotates first pulley 110 in the same direction as second pulley 120.

In this same embodiment of motion mechanism, second end 92 of elongated attachment member 90 is fixedly attached to circular belt 130 via attachment means. Attachment means may include any attachment means well known to those of ordinary skill in the art. Attachment member 90 has first end 91 and second end 92. First end 91 of attachment member 90 extends through slot 40. Object 100 attaches to first end 91 of attachment member 90. Object 100 can take many forms including, but not limited to, a ball, a toy or stuffed animal, a hand shape, any geometric shape, etc. as long as object 100 is of sufficient dimensions to allow a subject to easily follow it with his/her eyes.

Encoder 150 attaches to first pulley 110 and measures the position of object 100. In use, when object 100 is positioned at first end 41 of slot 40 then encoder 150 is “zeroed out” or “reset”. Object 100 moves in the direction of second end 42 of slot 40 until encoder 150 measures that object 100 is at the position of second end 42 of slot 40. At this position, a signal is transmitted from encoder 150 to controller 60 and microprocessor 140. Microprocessor 140 transmits a signal to controller 60 to generate the opposite voltage to motor 50 thus changing the direction of motor 50. This change in direction of motor 50 initiates the movement of object 100 towards first end 41 of slot 40. Once object 100 reaches first end 41 of slot 40, encoder 150 is again “zeroed out” and a signal is sent to controller 60 and microprocessor 140 which again changes the direction of motor 50 and thus also changes the direction of movement of object 100. An example of a type of encoder that can be used in the present invention is Phidget 3530.

In an alternative embodiment of pulley system, a plurality of stop mechanisms may be used instead of encoder 150 to change direction of object 100. A first stop mechanism may be positioned to the right of first pulley 110 and a second stop mechanism may be positioned to the left of second pulley 120. These positions would correspond to first end 41 of slot 40 and second end 42 of slot 40. Both stop mechanisms would be electrically connected to controller 60. Activation of stop mechanism would initiate movement of object 100 in the opposite direction. Stop mechanism may be any stop mechanism known to those of ordinary skill in the art. For example, stop mechanism may be pressure sensors, a laser mechanism, or a timing mechanism. In use, a pressure sensor would be activated once attachment member 90 was in a position to apply pressure to the pressure sensor. The pressure sensor would then send a signal to controller 60 to switch the voltage to the motor and thus change the direction of movement of object 100. If a laser mechanism were used, a laser beam would extend across circular belt 130 after first pulley 110 and before second pulley 120. Once attachment member 90 was in a position to break the beam of the laser, a signal would be sent to controller 60 to switch the voltage to the motor and thus change the direction of movement of object 100. If a timing mechanism were used, microprocessor 140 could send a signal at a predetermined time period to controller 60 to switch the voltage to the motor and thus change the direction of movement of object 100.

In an alternative embodiment of motion mechanism, a wheel with teeth system may be used as the motion mechanism. In this embodiment, a wheel with teeth is rotatably attached to a horizontal member that extends from first side 31 of housing 30 to second side 32 of housing 30. A motor would be fixedly attached to the wheel so that activation of the motor would cause the wheel (and the motor) to move horizontally across the horizontal member. Motor 50 is electrically connected to controller 60. An elongated attachment member 90 having a first end 91 and a second end 92 would be rotatably attached by its second end 92 to the wheel with teeth. First end 91 of attachment member 90 would extend through slot 40 and attach object 100. A first stop mechanism may be positioned at first end 41 of slot 40 and a second stop mechanism may be positioned at second end 42 of slot 40. Both stop mechanisms would be electrically connected to controller 60. Activation of stop mechanism would initiate movement of object 100 in the opposite direction. Stop mechanism may be any stop mechanism known to those of ordinary skill in the art. For example, stop mechanism may be pressure sensors, a laser mechanism, or a timing mechanism. In use, a pressure sensor would be activated once attachment member 90 was in a position to apply pressure to the pressure sensor. The pressure sensor would then send a signal to controller 60 to switch the voltage to the motor and thus change the direction of movement of object 100. If a laser mechanism were used, a laser beam would extend across circular belt 130 at first end 41 of slot 40 and at second end 42 of slot 40. Once attachment member 90 was in a position to break the beam of the laser, a signal would be sent to controller 60 to switch the voltage to the motor and thus change the direction of movement of object 100. If a timing mechanism were used, microprocessor 140 could send a signal at a predetermined time period to controller 60 to switch the voltage to the motor and thus change the direction of movement of object 100.

In use, motor 50 receives an activation signal from controller 60 which applies voltage to motor 50 and initiates movement of attachment member 90 (and thus object 100) horizontally across device 10. Once attachment member 90 reaches stop mechanism, a signal is sent to controller 60 to change the voltage to motor and thus change the direction of movement of object 100 to the opposite direction. These back and forth movements occur in cycles of 20, 30 or 40 to assist a subject in processing out negative emotions.

A further embodiment of motion mechanism includes a screw mechanism. IN this embodiment, an elongated screw is positioned within housing 30 extending from first end 31 of housing 30 to second end 32 of housing 30. The elongated screw would be attached to motor 50 at second end 32 of housing 30 and a rotatable component at first end 31 of housing 30. An elongated attachment member 90 having a first end 91 and a second end 92 would be rotatably attached by its second end 92 to the screw. First end 91 of attachment member 90 would extend through slot 40 and attach object 100. A first stop mechanism may be positioned at first end 41 of slot 40 and a second stop mechanism may be positioned at second end 42 of slot 40. Both stop mechanisms would be electrically connected to controller 60. Activation of stop mechanism would initiate movement of object 100 in the opposite direction. Stop mechanism may be any stop mechanism known to those of ordinary skill in the art. For example, stop mechanism may be pressure sensors, a laser mechanism, or a timing mechanism. In use, a pressure sensor would be activated once attachment member 90 was in a position to apply pressure to the pressure sensor. The pressure sensor would then send a signal to controller 60 to switch the voltage to the motor and thus change the direction of movement of object 100. If a laser mechanism were used, a laser beam would extend across circular belt 130 at first end 41 of slot 40 and at second end 42 of slot 40. Once attachment member 90 was in a position to break the beam of the laser, a signal would be sent to controller 60 to switch the voltage to the motor and thus change the direction of movement of object 100. If a timing mechanism were used, microprocessor 140 could send a signal at a predetermined time period to controller 60 to switch the voltage to the motor and thus change the direction of movement of object 100. Alternatively an encoder, similar to the encoder used in the embodiment of the pulley system, may be used to initiate the change of direction of the movement of the object.

In use, as motor 50 turns the screw in a first direction, the rotatably attached attachment member 90 moves horizontally in a first direction thus moving object 100 across the device in a first direction. Once the stop mechanism is activated, a signal is sent to controller 60 to switch the voltage to motor 50 and thus change the direction of movement for object 100.

Mechanical device 10 may include activation switch 160 which is electrically connected to controller 60. Activation switch 160 may be controlled by the client or the therapist. Activation switch 160 could have controls for modulating the speed of movement of object 100 as well as on/off power controls for device 10.

Alternatively, device 10 may be controlled by a remote control. If controlled by a remote control, the remote control would generally have means to start and stop the horizontal movement of object 100 as well as means for increasing or decreasing the movement speed of object 100.

Mechanical device 10 could also have a screen attached to second side 32 of housing 30 which could be extended upwards to block out any distractions behind device 10. In an embodiment, the screen is attached to a telescoping rod. The rod may be attached to second side 32 of housing 30. In its compressed position, the rod would not be visible from the front of device 10. In its extended position, the rod would extend upwards from second side 32 of housing 30 and be capable of remaining an extended position until the user physically urges the rod into the compressed position. This can be accomplished by any number of means known to those of ordinary skill in the art. In use, the screen would extend upwards and may stay in an upright position via an attachment means present on the upper end of the rod. Such attachment means may be a hook and loop system or any similar system.

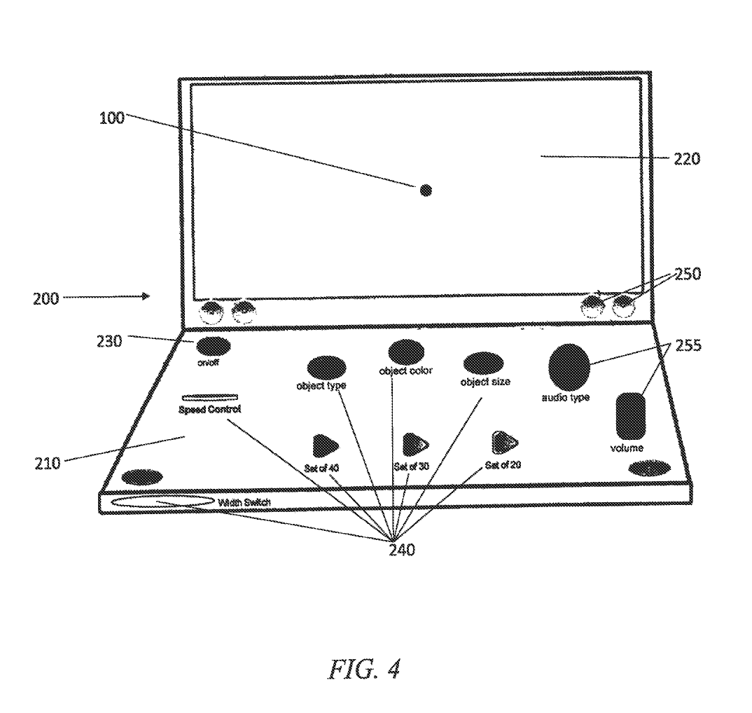

In a further embodiment as shown in FIG. 4, electronic device 200 is generally comprised of control component 210 having a front side 211, a back side 212, a top side 213 and a bottom side 214 and visual display component 220 having a front side 221 and a back side 222. Visual component 220 extends upwardly from back side 212 of control component 210, similar to a laptop computer. Visual component 220 may be comprised of a screen or other means which displays an object for the subject to follow with his/her eyes. The screen can be any screen which is capable of displaying a computer-generated object.

Power source 230 is electrically attached to control component 210. Power source 230 may be comprised of a battery that is housed within control component 210 or alternatively control component 210 may be plugged into a standard wall outlet.

Top side 213 of control component 210 contains a plurality of switches/buttons 240 which control the electrical operation of device 200 as well as the modes of operation of device 200. For example, switches 240 can be used to power on/off device 200; control the speed at which object 100 travels across visual component 220; control the type of object 100 that is displayed on visual component 220; control the size of object 100 displayed on visual component 220; control the color of object 100 displayed on visual component 220; control the shape of object 100 displayed on visual component 220; and track movement of object 100 displayed on visual component 220. With regard to speed, generally, the faster the object moves the better to aid in processing information. Clients may begin with a slower speed and increase it as they continue.

In addition, control component 210 of device 200 may include switches 240 to control the number of eye movements in each set. Switches 240 can be pre-programmed to a pre-determined number of eye movements. Alternatively there may be one switch that controls the number of eye movements. If one switch is used, the number of eye movements can be pre-programmed to the preferred number or the user may manually program the appropriate number of eye movements in each set. There is a preferred number of eye movements for adults (about 40 per set) and a different preferred number for children or those who process information very quickly (about 20 per set).

Device 200 is controlled by a microprocessor. This microprocessor may be pre-programmed with software for various modes of operation or may be hardwired for the various modes of operation.

In use, the device of the present invention (both device 10 and device 200) is positioned so that object 100 to is at eye level with the subject. The subject is instructed to stare straight ahead at object 100 and follow the movement of object 100 protruding from mechanical device 10 or alternatively on visual component 220 of electronic device 200 with his/her eyes. The preferred eye movement is horizontally from left to right and back again in a cycle. If used outside of therapy, the intent is that the subject would concentrate on any distressing sensations present in his/her body while following the object with only their eyes. It is important that the head of the subject is kept still and movement of object 100 is only followed by the eyes. One eye movement is calculated as one complete horizontal movement from left to right and back to the left.

The device tracks the correct amount of eye movements for each subject. After each set of eye movements, the subject should pause for a moment to ascertain whether the distressing sensations have moved, changed, or been eliminated. If the distressing sensations have not been modulated, the subject may continue with another set of eye movements. Means may be provided to continue with another set of eye movements such as a button, lever, switch, etc. that, when activated, alerts the device to begin tracking the eye movements again.

The correct amount of eye movements is based on the age of the subject and the rate at which the subject processes information. Beneficially, each set of eye movements can be between about 20 and 40 eye movements. For example, the device may have a button for about 40 eye movements which is the amount found to be beneficial for adults and teenagers. There may also be a button for about 20 eye movements which is the amount found to be beneficial for children who are pre-adolescent and process information quickly. There may also be a button for about 20 eye movements which is the amount found to be beneficial for younger children who require less eye movements to process sensations or other information while doing ART. A set of eye movements can have less than 20 or more than 40 eye movements per set in some instances. In these instances, the device can have a button which allows the subject to enter the number of eye movements needed as determined by the ART therapist.

The speed with which the object moves can be modulated by the subject or therapist. The optimum speed would be based on the speed at which the client processes. The minimum speed of the eye movements should not be less than about 40 movements per minute. The maximum amount of eye movements should not exceed about 180 movements per minute. A reasonable range of movements per minute is between about 80 and about 140 movements. The optimal number of movements is about 100 per minute. For eliminating negative images, the fastest speed a client can tolerate is best while for processing pleasant images, the speed may be lower.

In addition, the type of object that is tracked can be chosen from a pre-determined group of different objects. Object 100 can be any singular object that a subject can easily focus on. For example, object 100 may be a computer dot, a computer-simulated hand, a computer-simulated animal (for children), etc. for electronic-based device 200. Object 100 may be any physical object that is capable of being followed by the eyes including, but not limited to, a ball, a hand-shaped object, a stuffed animal such as a teddy bear, etc. for mechanical device 10. These objects can be switched out interchangeably. The size and color of object 100 can be modified by the user for electronic based device 200. The minimum size of object 100 is an object that is detectable by a subject having 20/20 vision at a reasonable distance from the subject. The maximum size of object 100 is that which is detectable in the field of vision of the subject. For example, if the device is two feet away from the subject then the maximum size of the object would be about six feet in diameter. For the mechanical device, a preferred size of object 100 is between about 6 inches and about 12 inches in diameter. For the electronic and computer software devices, a preferred size of object 100 is between about 1/16 of an inch and about 1 inch in diameter.

There is a minimum and a maximum distance that object 100 may travel for the therapy to be effective. The minimum distance that object 100 may travel is about 0.5 inches. The maximum distance object 100 may travel increases as the distance object 100 is positioned away from the subject increases. For example, if the device is two feet away from the subject then the maximum distance object 100 may travel is about six feet. The preferred distance object 100 may travel when using the mechanical device is between about 1 and about 2 feet. The preferred distance object 100 may travel when using the electronic or computer software based device is between about 2 inches and about 12 inches.

Speakers 250 may be included in control component 210 or visual component 220 of device 200 as well as volume switches 255 to control the volume of the audio. Device 200 can contain switches to activate voice instruction or “white noise” as well as switches that can produce audio and/or visual images to aid in sleep. Both electronic device 200 and mechanical device 10 can have a button that when activated allows the device to be used as a sleep aid. In use as a sleep aid, the device can have a pre-programmed voice activated guide that aids in relaxation and processing out the day's stress. The pre-programmed voice could explain what the client should be focused on and could instruct the client in relaxation exercises with the eye movements. Afterwards, the voice could guide the client through a positive guided imagery with eye movements. Electronic device 200 can also have voice-guided imagery that is presented on the screen to assist in inducing sleep. Both devices can be programmed to be shut off after a certain amount of time, similar to the “sleep” function on a television. In addition, both devices can be equipped with a button that when activated produces white noise. The white noise can be used in a therapy session to block out other noises.

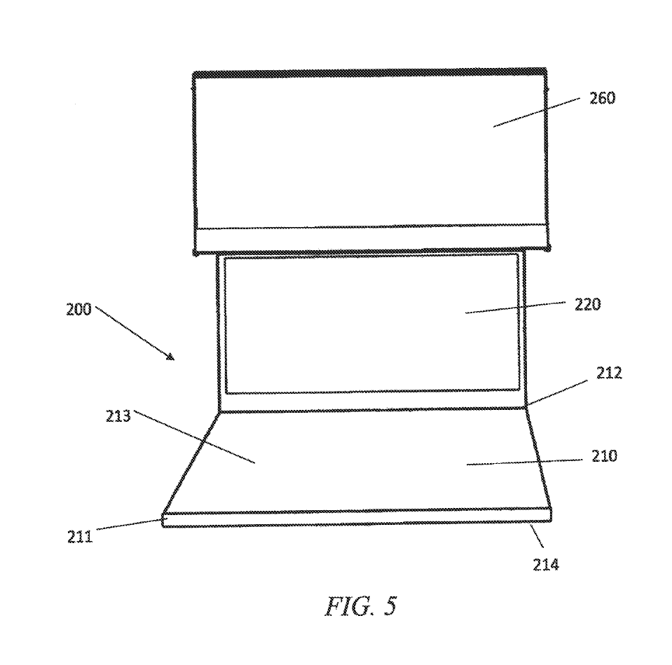

Screen 260 may extend upwards from visual component 220 or control component 210 to block out distractions during therapy as shown in FIG. 5.

Both embodiments of the device may be manually controlled or may be controlled remotely through the use of a remote control using infrared signals to communicate with controller.

The device of the instant invention can be used in conjunction with Accelerated Resolution Therapy (ART) to assist a client in processing through negative experiences in a short period of time. The examples below contain data supporting the use of ART to process through negative experiences such as post-traumatic stress disorder (PTSD).

### Example 1

16-consecutively-treated adult participants (age 21-57, mean: 42) from a study funded by the U.S. Substance Abuse and Mental Health Services Administration underwent between 2-5 sessions (median-3) of ART over a 2-week interval, AU participants had symptoms of psychological trauma, as indicated by presenting scores on the Psychiatric Diagnostic Screening Questionnaire (PDSQ) (range 41-73, median: 54) and PTSD Checklist-Civilian Version (PCL-C) (range 36-81, median: 52).

The PDSQ was used to screen for Axis I disorders and serve as a baseline assessment of psychopathology. This instrument has been validated against diagnostic criteria and interview-derived diagnoses over the course of 10 years and more than 3,000 administrations. (Zimmerman, M., & Chelminski, I. (2006). A scale to screen for DSM-IV Axis I disorders in psychiatric out-patients: performance of the Psychiatric Diagnostic Screening Questionnaire. Psychological Medicine, 36, 1601-1611; Zimmerman, M., & Mattia, J. I. (2001). A self-report scale to help make psychiatric diagnoses: the Psychiatric Diagnostic Screening Questionnaire. Archives of General Psychiatry, 58, 787-794). It can be quickly hand scored to obtain a total score (which functions as a global indicator of psychopathology) plus scale scores for 13 disorders: major depressive disorder, generalized anxiety disorder, panic disorder, posttraumatic stress disorder, alcohol abuse/dependence, drug abuse/dependence, psychosis, bulimia/binge-eating disorder, somatization disorder, obsessive-compulsive disorder, social phobia, hypochondriasis, and agoraphobia. Six items on the depression scale provide a measure of suicidal ideation. In general, a T-score of 40-60 is consistent with the level of symptomatology typically seen in the outpatient population, and indicates the presence of significant psychological distress (i.e. need for treatment).

The PCL-C Checklist is a self-administered 17-item scale (5-point scale for each item) that corresponds to key symptoms of PTSD. Reliability estimates range from 0.92 to 0.97, and the PCL has been validated in veterans. (Blanchard, E. B., et al., Psychometric properties of the PTSD checklist (PCL). Behavioral Research & Therapy, 1996. 34: p. 669-673; Weathers, F., et al., The PTSD Checklist (PCL): Reliability, Validity, and Diagnostic Utility. 1993: San Antonio, Tex.).

The ART sessions consisted of assessment of subjective units of distress on a 10-point scale; use of eye movements to process out anxiety and/or body sensations associated with recall of a traumatic memories; therapist directed implementation of a creative intervention to achieve “Voluntary Memory/Image Replacement” as defined previously; and closure assessment. The closure assessment is an assessment by the therapist in conjunction with the subject as to whether or not the traumatic experience/image that has been causing distress has been successfully treated.

All participants were Caucasian, 13 (81.2%) were female, and 3 (18.7%) were Hispanic. Before and after ART, mean scores on self-report instruments were as follows: PCL-C (54.1±13.1 vs. 31.4±10.9: p<0.0001); CES-D (25.9±8.1 vs. 16.4±4.6: p<0.0001); and Brief Symptom Inventory (BSI) (24.7±13.3 vs. 9.1±8.1: p<0.0001).

The Brief Symptom Inventory (BSI) contains 18-items and is a shortened version of the Symptom Checklist 90-Revised (SCL-R-90) designed to measure clinically relevant psychological symptoms in adolescents and adults. (Derogatis, 2001) This instrument encompasses 3 factors: Somatization, Depression, and Anxiety, along with a Global Severity Index. This inventory presents satisfactory reliability indexes, both for the individual factors (ranging from 0.74 to 0.84) and the Global Severity Index (0.89).

The Centers for Epidemiological Studies Depression Scale (CES-D) is a widely used 20-item self-report scale which measures the current level of depressive symptomatology in the general population, with an emphasis on depressed mood during the past week (Radloff, 1977). The CES-D incorporates the main symptoms of depression and was derived from five validated depression scales including the Beck Depression Inventory (BDI). It is freely available in the public domain, has been validated in community and primary care populations, in cardiac patients (Penninx et al., 2001) and older populations (Zich et al., 1990), and has good test-retest reliability (Ensel, 1986). Reliability estimates range from 0.84 to 0.92

Over a median of 3 ART sessions, large, clinically-significant improvements in symptoms of psychological trauma and depression were observed. ART appears to achieve rapid resolution of symptoms of psychological trauma.

### Example 2

Example 2 uses ART and incorporates the use of eye movements administered in a brief treatment period, defined as about 1-5 one-hour sessions within about 3 weeks, to resolve post-traumatic stress disorder (PTSD). Eighty adults, ages 21-60 years, with symptoms of PTSD were recruited from the Tampa Bay area. The ART-based psychotherapy was designed to minimize anxiety and body sensations associated with recall of traumatic memories and to replace distressing images with favorable ones. Participants' mean age was 40 years, 77% were female, and 29% were Hispanic. Participants underwent a median of 3 ART sessions, 66 of 80 (82.5%) completed treatment, and 52 of 66 (78.8%) provided 2-month follow-up data. Mean scores pre- and post-ART and at 2-month follow-up were assessed as follows: PTSD Checklist: 54.1±12.3 vs. 31.3±11.5 vs. 29.5±12.0; Brief Symptom Inventory: 30.1±14.2 vs. 10.1±10.8 vs. 9.6±11.9; Center for Epidemiologic Studies Depression Scale: 29.0±10.7 vs. 11.8±11.1 vs. 13.4±12.1; Trauma Related Growth Inventory-Distress scale: 18.7±4.0 vs. 7.4±5.9 vs. 8.0±5.9 (p<0.0001 for all pre-ART vs. post-ART and 2-month comparisons). No serious adverse events were reported thus it was concluded that ART appears to be a brief, safe, and effective treatment for symptoms of PTSD.

### Methods

Participant inclusion criteria were: (i) between the age of 21-60 years inclusive; (ii) symptoms indicative of PTSD, as defined as a score of >40 on the PTSD Checklist (range 17-85)—Civilian version (PCL-C) (Blanchard, E. B., Jones-Alexander, J., Buckley, T. C., & Forneris, C. A. (1996). Psychometric properties of the PTSD checklist (PCL). Behavioral Research & Therapy, 34, 669-673; Weathers, F., Litz, B., Herman, D., Huska, J., & Keane, T. (1993). The PTSD Checklist (PCL): Reliability, Validity, and Diagnostic Utility Paper presented at the Annual Convention of the International Society for Traumatic Stress Studies, San Antonio, Tex. San Antonio, Tex.), or in the absence of a score >40, other documented evidence of symptoms, including a high PTSD subscale score and/or endorsement of specific PTSD item responses on the Psychiatric Diagnostic Screening Questionnaire (PDSQ); (iii) ability to read and speak English to complete survey questions; and (iv) denial of suicidal and homicidal ideation or intent and no evidence of psychotic behavior or otherwise being in psychological crisis.

Exclusion criteria were: (i) brain injury prohibiting speech, writing, and purposeful actions; (ii) current suicidal ideation; (iii) major psychiatric disorder primary to symptoms of psychological trauma; (iv) current treatment for substance abuse; (v) previous diagnosis of eye movement disorder anticipated to interfere with treatment (e.g. amblyopia); and (vi) any medical condition that, in the judgment of the Principal Investigator and/or ART therapist, may place the individual at high risk due to a potential heightened emotional reaction (e.g. previous heart attack, seizure disorder). Individuals with previous treatment for symptoms of PTSD, yet with residual symptoms that met inclusion criteria upon screening, were eligible for the study.

### Recruitment

Participant recruitment occurred through caseload referrals from licensed mental health therapists in the study area, flyers and brochures, direct meetings with providers of mental health services, and as a result of local media coverage of ART. All participants were treated at the USF College of Nursing and received $20 for completion of self-report questionnaires (see below) before and after treatment and at 2-month follow-up. The study protocol was approved by the Institutional Review Board and all participants provided written informed consent.

### Therapist Training

All therapists underwent intensive training in ART conducted in person by the developer (L.R.) and lead trainer (A.S.) and in accordance with the ART training manual. This included two 8-hour days on the theory, principles, and protocol for conducting ART including intake assessment, intervention techniques, eye movement regimen, challenges and solutions, and closure techniques. This was followed by directly-observed supervised practice, and then follow-up training and assessment. A total of 8 therapists conducted the ART sessions including: 1 Clinical Psychologist (Psy.D.); 4 licensed mental health counselors (LMHC); 2 licensed clinical social workers (LCSW); and 1 licensed marriage and family therapist (LMFT). Therapists included males and females and those of Hispanic and non-Hispanic ethnicity.

### Participant Intake Assessment

Participants completed 3 screening instruments as part of their intake assessment. This included the 17-item PCL-C Checklist (Blanchard, E. B., Jones-Alexander, J., Buckley, T. C., & Forneris, C. A. (1996). Psychometric properties of the PTSD checklist (PCL). Behavioral Research & Therapy, 34, 669-673; Weathers, F., Litz, B., Herman, D., Huska, J., & Keane, T. (1993). The PTSD Checklist (PCL): Reliability, Validity, and Diagnostic Utility Paper presented at the Annual Convention of the International Society for Traumatic Stress Studies, San Antonio, Tex. San Antonio, Tex.), self-developed 9-item ART Intake Questionnaire, and the 125-item (yes/no) PDSQ.

The PDSQ was used to screen for Axis I disorders and serve as a baseline assessment of psychopathology. This instrument has been validated against diagnostic criteria and interview-derived diagnoses over the course of 10 years and more than 3,000 administrations. (Zimmerman, M., & Chelminski, I. (2006). A scale to screen for DSM-IV Axis I disorders in psychiatric out-patients: performance of the Psychiatric Diagnostic Screening Questionnaire. Psychological Medicine, 36, 1601-1611; Zimmerman, M., & Mattia, J. I. (2001). A self-report scale to help make psychiatric diagnoses: the Psychiatric Diagnostic Screening Questionnaire. Archives of General Psychiatry, 58, 787-794). It can be quickly hand scored to obtain a total score (which functions as a global indicator of psychopathology) plus scale scores for 13 disorders: major depressive disorder, generalized anxiety disorder, panic disorder, posttraumatic stress disorder, alcohol abuse/dependence, drug abuse/dependence, psychosis, bulimia/binge-eating disorder, somatization disorder, obsessive-compulsive disorder, social phobia, hypochondriasis, and agoraphobia. Six items on the depression scale provide a measure of suicidal ideation.

In general, a T-score of 40-60 is consistent with the level of symptomatology typically seen in the outpatient population, and indicates the presence of significant psychological distress (i.e. need for treatment). “Screen fail” participants (i.e. those not meeting enrollment criteria, including those with a T-score >70 indicating need for a higher level of care) were offered a direct referral for counseling in the community, or 2 complimentary sessions at the study site (and appropriate referral thereafter) of an empirically-based method of psychotherapy.

### ART Protocol

The ART protocol is theoretically grounded in and uses cognitive behavioral and experiential therapies including Gestalt and Guided Imagery, along with psychodynamic psychotherapy. Table 1 lists primary components of the ART protocol and their relationship to established psychotherapies. Unique features of ART include: (i) a central focus on reconsolidating disturbing memories; (ii) a fixed number of eye movements (sets of 40) to reduce physiological and affective responses during focused recall of events; (iii) continuous assessment of physiological sensations and images targeted for cognitive reduction/removal; and (iv) patient directed re-envisioning of events in a resolving narrative with Voluntary Image Replacement (VIR). A description of the initial conceptual development of ART has been published in Rosenzweig, L. (2010). The development of Accelerated Resolution Therapy (ART): insights, examples, and applications. International Journal of Trauma Professionals 3, 1-9, which is hereby incorporated in its entirety into this disclosure.

TABLE 1
Primary Components of the ART Protocol and Relationship to
Established Psychotherapies

| | Cognitive Behavioral Therapies | | | | |
|---|---|---|---|---|---|
| | | | | Eye | |
| | | | | Movement | |
| | | Guided | Exposure | e.g. | Psycho- |
| Component of ART Protocol | Gestalt | Imagery | Therapy | EMDR | Dynamic |
| Bilateral stimulation by use of eye | | | | X | |
| movements | | | | | |
| Purposeful recall of traumatic experience(s) | | | X | X | |
| Identify earlier time in life if (when) similar | | | | | X |
| sensations were experienced (i.e. use of | | | | | |
| “Scene Match” technique) | | | | | |
| Focus on sensations in the moment, and | X | | | X^{a} | |
| continuous assessment of sensations | | | | | |
| following individual sets of eye movement | | | | | |
| Resolving narrative and replace distressing | | | | | |
| image(s) with positive image(s) (i.e. | | | | | |
| Voluntary Image Replacement (VIR) by use | | | | | |
| of “Director” intervention) | | | | | |
| “Speak” to parts of one's past (e.g. say | X | X | | | |
| goodbye to someone lost to resolve past | | | | | |
| issues) | | | | | |
| Use of metaphors for problem solving | | X | | | |
| Reinforce new image/scene is visualized and | | | | | |
| original image/scene is replaced (i.e. use of | | | | | |
| “Transfer” intervention) | | | | | |
| Identify future events likely to occur which | | X | | | |
| may have formerly triggered the unpleasant | | | | | |
| sensations, and conduct “target trigger | | | | | |
| times” protocol if needed | | | | | |

^{a}ART continuously assesses sensations after each set of 40 eye movements, whereas EMDR does not require a fixed amounts of eye movements or sensation checks after each set.

### Individual ART Sessions

Consenting participants underwent between about 1-5 sessions of ART (within about 3 weeks), each approximately 60 to 75 minutes in length. In brief, the 2 major components of each ART session were: (i) minimize or eliminate physiological response associated with recall of the traumatic memory(ies); and (ii) re-envisioning of events with the VIR technique. Thus, individual sessions included: (i) initial assessment of Subjective Units of Distress (SUDS) on a 10-point scale; (ii) discussion with the participant on the intended use of the VIR for the distressing scene(s) to be treated; (iii) with use of lateral eye movements, reduction or elimination of anxiety and/or somatic sensations associated with purposeful recall of the traumatic memory(ies); (iv) therapist-directed implementation of a creative intervention(s) (from the ART manual) to achieve the VIR; (v) attempted recall of the original distressing scene versus the new scene to assess participant response to the VIR; (vi) therapist-directed discussion about any future events anticipated to trigger the symptoms associated with the original memory and, if found, implementation of an ART intervention to neutralize their potential potency; (vii) closure assessment, to include discussion of any future traumatic memories to be treated in subsequent ART sessions; and (viii) session closeout assessment of SUDS on 10-point scale. The therapist, in consultation with the participant, determined the total number of ART sessions based on the initial intake assessment, including assessment of the number of ART scenes needing to be resolved, and session-by-session response to therapy.

### Data Collection

After written consent and clinical screening for determination of study eligibility, participants completed a self-developed demographic and brief medical history questionnaire. Baseline completion of self-reported outcome measures (in addition to the previously completed PCL-C) included the following measures: (1) 18-item Brief Symptom Inventory (BSI) (Derogatis, L. R. 2001. Brief Symptom Inventory (BSI)-18. Administration, scoring and procedures manual. Minneapolis, Minn.); (2) 20-item Center for Epidemiologic Studies Depression Scale (CES-D) (Radloff, L. S. (1977). The CES-D scale: A self-report depression scale for research in the general population. Applied Psychological Measurement, 1, 385-401); (3) 21-item State-Trait Inventory for Cognitive and Somatic Anxiety (STICSA) (Ree, M. J., French, D., MacLeod, C., & Locke, V. (2008). Distinguishing cognitive and somatic dimensions of state and trait anxiety: Development and validation of the state-trait inventory for cognitive and somatic anxiety (STICSA). Behavioural and Cognitive Psychotherapy, 36, 313-332); (4) 26-item Self-Compassion Scale (SCS) (Neff, K. D. (2003). The development and validation of a scale to measure self-compassion. Self and Identity, 2, 223-250); (5) 29-item Aggression Questionnaire (AQ) (Buss, A. H., & Perry, M. (1992). The aggression questionnaire. Journal of Personality and Social Psychology, 63, 452-459); (6) 10-item Alcohol Use Disorder Identification Test (AUDIT) (Saunders, J. B., Aasland, O. G., Babor, T. F., de la Fuente, J. R., & Grant, M. (1993). Development of the Alcohol Use Disorders Identification Test (AUDIT): WHO collaborative project on early detection of persons with harmful alcohol consumption. II. Addiction, 88, 791-804); (7) 32-item Trauma-Related Guilt Inventory (TRGI) (Kubany, E. S. (1996). Development and validation of the Trauma-Related Guilt Inventory (TRGI). Psychological Assessment, 8, 428-444); (8) 21-item Post-Traumatic Growth Inventory (PTGI) (Tedeschi, R. G., & Calhoun, L. G. (1996). The Posttraumatic Growth Inventory: measuring the positive legacy of trauma. Journal of Traumatic Stress, 9, 455-471); and (9) the Pittsburgh Sleep Quality Index (PSQI) (Buysse, D. J., Reynolds, C. F., Monk, T. H., Berman, S. R., & Kupfer, D. J. (1989). The Pittsburgh Sleep Quality Index: A new instrument for psychiatric practice and research. Psychiatric Residency, 28, 193-213).

### Brief Symptom Inventory (BSI)

The 18-item BSI (Derogatis, 2001) is a shortened version of the Symptom Checklist 90-Revised (SCL-R-90) designed to measure clinically relevant psychological symptoms in adolescents and adults. This instrument encompasses 3 factors: Somatization, Depression, and Anxiety, along with a Global Severity Index. This inventory presents satisfactory reliability indexes, both for the individual factors (ranging from 0.74 to 0.84) and the Global Severity Index (0.89).

### Centers for Epidemiological Studies Depression Scale (CES-D)

The CES-D is a widely used 20-item self-report scale which measures the current level of depressive symptomatology in the general population, with an emphasis on depressed mood during the past week (Radloff, 1977). The CES-D incorporates the main symptoms of depression and was derived from five validated depression scales including the Beck Depression Inventory (BDI). It is freely available in the public domain, has been validated in community and primary care populations, in cardiac patients (Penninx et al., 2001) and older populations (Zich et al., 1990), and has good test-retest reliability (Ensel, 1986). Reliability estimates range from 0.84 to 0.92.

### State-Trait Inventory for Cognitive and Somatic Anxiety (STICSA)

The 21-item State-Trait Inventory for Cognitive and Somatic Anxiety (STICSA; Ree, MacLeod, French, & Locke, 2000) was designed to assess cognitive and somatic symptoms of anxiety as they pertain to one's mood in the moment (state) and in general (trait). The instrument items take an estimated 6 minutes to complete. The STICSA is a reliable and valid measure of state and trait cognitive and somatic anxiety, with coefficients ranging from 0.87 to 0.90.

### Self-Compassion Scale (SCS)

The SCS is a 26-item measure assessing personal levels of self-compassion developed by Neff (2003). The measure consists of six different subscales that assess kindness, self-judgment, common humanity, isolation, mindfulness, and over-identification. Time to complete the scale is estimated to be approximately 6 to 10 minutes. The SCS shows good construct validity and good test-retest reliability (r=0.93; Neff, 2003).

### Aggression Questionnaire (AQ)

The Aggression Questionnaire (AQ) (Buss & Perry, 1992) is a widely used measure assessing hostility and aggression. This 29-item questionnaire takes approximately 10 minutes to complete and is written at a third-grade reading level. The AQ items describe various characteristics related to aggression. The respondent simply rates each item on a 5-point scale ranging from “Not at all like me” to “Completely like me.” Reliability coefficients range from 0.77 to 0.85.

### Alcohol Use Disorder Identification Test (AUDIT-C)

The AUDIT-C is an effective, brief screening test that consists of 10 alcohol consumption questions that measure heavy drinking and/or active alcohol abuse or dependence. Each item is rated on a 5-point scale measuring alcohol frequency or consumption, such as: Never (0), Monthly or less (1), 2-4 times a month (2), 2-3 times a week (3), or 4 or more times a week (4). The AUDIT was developed by the World Health Organization (WHO) as a simple method of screening for excessive drinking and to assist in brief assessment (Saunders, 1993a; Saunders, 1993b). Internal consistency reliability has been reported as 0.80.

### Trauma Related Guilt Inventory (TRGI)

The TRGI (Kubany, 1996) is a 22-item measure that assesses event-focused trauma-related guilt. The inventory has high internal consistency and adequate temporal stability, and its scales and subscales significantly correlate with measures of guilt and posttraumatic stress disorder, depression and adjustment. Reliability coefficients range from 0.73 to 0.91. Time to complete the inventory is estimated to be 6 minutes.

### Posttraumatic Growth Inventory (PTGI)

This is a 21-item measure that assesses positive changes that may follow traumatic events. The scale includes factors of New Possibilities, Relating to Others, Personal Strength, Spiritual Change, and Appreciation of Life and is estimated to take 6 to 10 minutes to complete. The scale appears to have utility in determining the success of individuals, in reconstructing or strengthening their perceptions of self, others, and the meaning of events during the aftermath of trauma. Reliability coefficients have reported to be 0.90.

### Pittsburgh Sleep Quality Index (PSQI)

This 15-item self-report developed in 1989 (Buysse et al., 1989) measures the quality and patterns of sleep in adults. It is estimated to take 3 to 6 minutes to complete. A global PSQI score greater than 5 yielded a diagnostic sensitivity of 89.6% and specificity of 86.5% (kappa=0.75, p less than 0.001) in distinguishing good and poor sleepers. Internal consistency reliability has been estimated to range from 0.77 to 0.81.

### State-Trait Anxiety Inventory (STAI)

The STAI, developed by Spielberger (1983) is a widely used 40-item measure in which items are rated on a 1-4 Likert scale. Twenty items assess State anxiety (STAI-S) and 20 items assess Trait anxiety (STAI-T). Psychometric properties are well-established (Spielberger, 1983), with reliability coefficients ranging from 0.73 to 0.86. Higher scores indicate greater levels of state and trait anxiety. Low level stability for the State-anxiety scale is expected since responses to the items on this scale are thought to reflect the influence of existing transient situational factors at the time of testing. Time to complete the STAI is 10 minutes.

These measures were selected to assess a wide range of psychological treatment response and on the basis of established reliability and validity. The outcome measures were completed immediately before the first ART session, after the final ART session, and at 2 months post-treatment. Occurrence of adverse events was inquired by the treating therapist prior to each ART session including the nature and intensity of each event, subsequent treatment actions, and judgment as to whether the event was causally related to use of ART.

### Statistical Methods

Continuous variables are expressed as mean±standard deviation (SD); categorical variables are presented as percentages. Paired t tests were used to compare changes in the above defined outcome measures before and after treatment completion and at 2-month follow-up. Standardized effect sizes for outcome measures were calculated as: ((mean before ART−mean after ART)/standard deviation of treatment difference scores). (Morris, S. B., & DeShon, R. P. (2002). Combining effect size estimates in meta-analysis with repeated measures and independent-groups designs. Psychological Methods, 7, 105-125).

McNemar's test for paired data was used to compare the proportion of subjects above established clinical cutoff scores for PTSD, depression, and sleep dysfunction before and after treatment with ART and at 2-month follow-up. In sensitivity analyses, analysis of covariance (ANCOVA) was used to compare initial treatment response between participants with and without 2-month follow-up data. Due to multiple comparisons, a two-tailed p-value of <0.01 was used to define statistical significance.

### Results

A total of 97 persons were screened, of whom, 17 (17.5%) were determined to be ineligible for the study. Primary reasons for ineligibility included major concomitant psychiatric disorder primary to psychological trauma or being in psychological crisis (n=11), and subclinical levels of symptomatology of psychological trauma (n=4) (FIG. 1). Among the 80 enrolled participants, 66 (82.5%) completed the full course of treatment including initial post-treatment assessment. The median number of ART sessions held was three for participants enrolled and four for those enrolled and who completed treatment. Principal reasons identified for non-completion were: (i) logistical and scheduling conflicts (n=3); (ii) advice/interaction by another mental health professional (n=2); difficulty in performing eye movements (n=1, participant had a history of surgically-corrected amblyopia); and other/unspecified reasons (n=8). None of the 14 participants who did not complete treatment specified the occurrence of adverse effects. Among the 66 participants who completed the full course of treatment, 52 completed 2-month follow-up assessments and 14 were lost to follow-up.

The mean age of study participants was 40.0±10.2 years, 77% were female, 89% were of Caucasian race, and 29% were of Hispanic ethnicity (Table 2). The mean PDSQ T-score at study entry was 53.7±8.6 indicating, on average, clinically significant psychopathology, as well as high PTSD symptomatology as evidenced by a mean PCL-C score of 54.5±13.0. Eleven participants (13.7%) had PCL-C scores below 40 yet were enrolled in the study based on their PTSD subscale score on the PDSQ meeting the cutoff score of ≧5 (10 of 11 participants), and all 11 participants endorsing at least 1 of the “critical” DSM-IV PTSD items.

Of note, approximately half of all participants reported ≧5 traumatic memories still impacting their life, 80% reported living with traumatic memories for ≧7 years. The trauma(s) for which treatment was sought was classified as experiencing violent or abusive crime (51%), loss of a loved one (29%), divorce (11%), chronic or acute illness (10%), and other. Medication use at study entry was common and highly variable. This included current use of anti-depressant/anti-anxiety medications (29% of all participants); medications for pain (11%); sleep medications (8%), psychoactive medications (5%); and ADD/ADHD medications (5%).

TABLE 2
Demographics and Presenting Characteristics

| | All | Male | Female |
|---|---|---|---|
| Characteristic | (n = 80) | (n = 18) | (n = 62) |
| Age in years (mean ± SD) | 40.0 ± 10.2 | 41.9 ± 11.8 | 40.6 ± 9.8 |
| Race (%) | | | |
| Black or African American | 7.5 | 0.0 | 9.7 |
| White | 88.8 | 94.4 | 87.1 |
| Asian | 1.3 | 0.0 | 1.6 |
| Native Hawaiian/Pacific Islander | 0.0 | 0.0 | 0.0 |
| American Indian/Alaskan Native | 1.3 | 0.0 | 1.6 |
| Not reported | 1.2 | 5.6 | 0.0 |
| Hispanic (%) | 28.6 | 5.6 | 35.6 |
| Married (%) | 58.8 | 61.1 | 58.1 |
| Employed - full or part time (%) | 59.8 | 72.2 | 55.9 |
| PDSQ score (mean ± SD) (T-score) | 53.7 ± 8.6 | 54.6 ± 8.6 | 53.5 ± 8.6 |
| Less than 40 (%) | 2.5 | 0.0 | 3.2 |
| 40 to 60 (%) | 75.0 | 77.8 | 74.2 |
| More than 60 (%) | 22.5 | 22.2 | 22.6 |
| PCL-C score (mean ± SD) | 54.5 ± 13.0 | 57.3 ± 14.3 | 53.7 ± 12.6 |
| Less than 40 (%) | 13.8 | 11.1 | 14.5 |
| 40 to 60 (%) | 47.5 | 38.9 | 50.0 |
| More than 60 (%) | 38.8 | 50.0 | 35.5 |
| No. of traumatic memories still impacting life (%) | | | |
| 1 to 2 | 19.0 | 23.57 | 17.7 |
| 3 to 4 | 31.6 | 35.3 | 30.6 |
| 5 or more | 49.4 | 41.2 | 51.6 |
| Previous treatment for PTSD/other MH condition | 67.9 | 50.0 | 73.3 |
| (%) | | | |
| On disability for PTSD or other MH disorder (%) | 10.1 | 5.9 | 11.3 |
| Guilt associated with traumatic memory(ies) (%) | 81.3 | 88.9 | 79.0 |
| Time lived with traumatic memory(ies) (%) | | | |
| Less than 1 year | 6.3 | 5.6 | 6.5 |
| 1 to 6 years | 13.8 | 22.2 | 11.3 |
| 7 years or more | 80.0 | 72.2 | 82.3 |

PDSQ: Psychiatric Diagnostic Screening Questionnaire.
PCL-C: PTSD Checklist, Civilian Version.
MH: Mental Health

For the 52 subjects who completed treatment and had 2-month follow-up data, very large, statistically significant initial treatment effects were observed across a range of symptom measures (Table 3, left side). For the primary outcome of PTSD symptoms, the mean score on the PCL-C was 54.1±12.2 before ART versus 31.3±11.5 after ART (mean difference=22.8±13.5; effect size=1.69; p<0.0001). In rank order, the largest effect sizes observed were for the following measures: TRGI-Distress subscale (effect size=1.89), BSI (effect size=1.72), PCL-C (effect size=1.69), STICSA-Cognitive Anxiety subscale (effect size=1.59), and CES-D (effect size=1.38) (p<0.0001 for all measures). Large clinical improvements in symptoms were also reported for all subscales of the PTGI, STICSA-Somatic Anxiety subscale, PSQI, TRGI global guilt and guilt cognition subscales, and SCS (p<0.0001 for all measures).

TABLE 3
Self-Report Changes in Symptoms Before and After Treatment with ART

| | | | | | | | Pre-Treatment Versus 2-Month | | | | | |
|---|---|---|---|---|---|---|---|---|---|---|---|---|
| Symptom | Pre-Versus Post-Treatment | | | | | | Follow-Up | | | | | |
| Measure | N | Pre^{a} | Post^{a} | Diff^{ab} | ES | P | N | Pre^{a} | 2-Mo.^{a} | Diff^{ab} | ES | P |
| PTSD | 52 | 54.1 | 31.3 | 22.8 | 1.69 | <0.0001 | 52 | 54.1 | 29.5 | 24.5 | 2.01 | <0.0001 |
| Checklist | | (12.2) | (11.5) | (13.5) | | | | (12.2) | (12.0) | (12.2) | | |
| (PCL-C) | | | | | | | | | | | | |
| Brief | 50 | 30.1 | 10.1 | 20.0 | 1.72 | <0.0001 | 52 | 30.1 | 9.6 | 20.5 | 1.54 | <0.0001 |
| Symptom | | (14.2) | (10.8) | (11.6) | | | | (14.2) | (11.9) | (13.3) | | |
| Inventory | | | | | | | | | | | | |
| CES-D | 52 | 29.0 | 11.8 | 17.2 | 1.38 | <0.0001 | 51 | 29.0 | 13.3 | 15.5 | 1.41 | <0.0001 |
| (Depression) | | (10.7) | (11.1) | (12.4) | | | | (10.7) | (12.1) | (11.0) | | |
| STICSA | 52 | 20.6 | 13.8 | 6.8 | 1.08 | <0.0001 | 52 | 20.6 | 14.8 | 5.7 | 1.10 | <0.0001 |
| (Somatic) | | (7.0) | (3.5) | (6.2) | | | | (7.0) | (5.1) | (5.2) | | |
| STICSA | 52 | 25.0 | 15.5 | 9.5 | 1.59 | <0.0001 | 52 | 25.0 | 17.4 | 7.6 | 1.13 | <0.0001 |
| (Cognitive) | | (6.6) | (5.0) | (6.0) | | | | (6.6) | (6.8) | (6.7) | | |
| Pittsburgh | 48 | 9.8 | 6.4 | 3.4 | 1.01 | <0.0001 | 47 | 9.8 | 6.2 | 3.6 | 0.89 | <0.0001 |
| Sleep Quality | | (4.6) | (4.4) | (3.4) | | | | (4.6) | (4.2) | (4.0) | | |
| Trauma | | | | | | | | | | | | |
| Related | | | | | | | | | | | | |
| Growth | | | | | | | | | | | | |
| Global | 52 | 4.6 | 1.6 | 3.0 | 1.25 | <0.0001 | 52 | 4.6 | 1.9 | 2.7 | 0.98 | <0.0001 |
| Guilt | | (2.4) | (1.9) | (2.4) | | | | (2.4) | (2.0) | (2.8) | | |
| Distress | 52 | 18.7 | 7.4 | 11.2 | 1.89 | <0.0001 | 52 | 18.7 | 8.0 | 10.7 | 1.89 | <0.0001 |
| | | (4.0) | (5.9) | (5.9) | | | | (4.0) | (5.9) | (5.6) | | |
| Guilt | 52 | 44.3 | 24.1 | 20.2 | 1.17 | <0.0001 | 52 | 44.3 | 24.4 | 19.9 | 1.04 | <0.0001 |
| Cognition | | (18.3) | (13.2) | (17.3) | | | | (18.3) | (14.9) | (19.2) | | |
| Post- | | | | | | | | | | | | |
| Traumatic | | | | | | | | | | | | |
| Growth | | | | | | | | | | | | |
| I: Relation | 51 | 12.1 | 17.0 | 4.9 | 0.89 | <0.0001 | 51 | 12.1 | 15.3 | 3.2 | 0.66 | <0.0001 |
| to Others | | (6.8) | (6.6) | (5.5) | | | | (6.8) | (6.9) | (4.9) | | |
| II: New | 51 | 12.6 | 16.9 | 4.3 | 0.71 | <0.0001 | 51 | 12.6 | 15.4 | 2.8 | 0.50 | 0.0009 |
| Possibilities | | (6.5) | (6.6) | (6.1) | | | | (6.5) | (7.1) | (5.5) | | |
| III: | 51 | 8.4 | 11.8 | 3.4 | 0.61 | <0.0001 | 51 | 8.4 | 11.5 | 3.1 | 0.67 | <0.0001 |
| Personal | | (5.4) | (6.2) | (5.6) | | | | (5.4) | (5.5) | (4.6) | | |
| Strength | | | | | | | | | | | | |
| IV: | 51 | 5.2 | 7.2 | 2.0 | 0.74 | <0.0001 | 51 | 5.2 | 6.4 | 1.2 | 0.39 | 0.008 |
| Spiritual | | (2.8) | (2.6) | (2.7) | | | | (2.8) | (3.0) | (3.1) | | |
| Change | | | | | | | | | | | | |
| V: | 51 | 7.6 | 9.6 | 1.9 | 0.44 | 0.0004 | 51 | 7.6 | 9.2 | 1.6 | 0.39 | 0.009 |
| Appreciation- | | (3.9) | (4.2) | (4.3) | | | | (3.9) | (3.9) | (4.0) | | |
| Life | | | | | | | | | | | | |
| Self- | 52 | 67.2 | 84.4 | 17.3 | 0.85 | <0.0001 | 52 | 67.2 | 82.0 | 14.8 | 0.83 | <0.0001 |
| Compassion | | (16.8) | (21.4) | (20.3) | | | | (16.8) | (21.1) | (17.8) | | |
| Scale | | | | | | | | | | | | |
| Aggression | 51 | 76.3 | 63.9 | 12.3 | 0.96 | <0.0001 | 51 | 76.3 | 64.0 | 12.3 | 0.89 | <0.0001 |
| Questionnaire | | (20.1) | (20.1) | (12.8) | | | | (20.1) | (21.2) | (13.8) | | |
| Alcohol Use | 50 | 3.0 | 2.5 | 0.5 | 0.27 | 0.03 | 52 | 3.0 | 1.8 | 1.2 | 0.50 | 0.0008 |
| (AUDIT) | | (3.3) | (3.0) | (1.8) | | | | (3.3) | (2.1) | (2.2) | | |

^{a}Presented as mean (standard deviation).
^{b}All mean differences are coded with positive numbers reflecting improvements in symptoms. ES: effect size. All comparisons of symptom measure scores at post-treatment versus 2-month follow-up were not statistically significant at P < 0.01 (specified level for statistical testing) with the following exceptions: STICSA-Cognitive (p = 0.008); Alcohol Use-AUDIT (p = 0.006).

In examining sustainability of treatment effects, symptom measure scores at 2 month follow-up were essentially parallel to those reported after treatment completion (Table 3, right side). In rank order, the largest pre-treatment to 2-month follow-up effect sizes observed were for the following measures: PCL-C (effect size=2.01), TRGI-Distress subscale (effect size=1.89), BSI (effect size=1.54), and CES-D (effect size=1.41) (p<0.0001 for all measures). Importantly, symptom scores did not differ statistically (at p<0.01) between any of the initial post-treatment versus 2-month follow-up assessments with the exception of the STICSA-Cognitive Anxiety subscale score being nominally higher at 2 months (15.5±5.0 versus 17.4±6.8; effect size=0.36, p=0.01), and conversely, alcohol use as measured by the AUDIT being nominally lower at 2 months (2.5±3.0 versus 1.8±2.1; effect size=0.46, p=0.005). Thus, results obtained post-treatment and at 2-month follow-up can be interpreted as approximately equivalent.

In subgroup analyses by age (<40 vs. ≧40 years), number of reported traumas still impacting life (<5 vs. ≧5), presenting PCL-C score (<50 vs. ≧50), and presenting PDSQ-T score (<50 vs. ≧50), large, clinically significant reductions in symptoms of PTSD were consistently reported after treatment completion and at 2-month follow-up (FIG. 2 p<0.001 for all comparisons with pre-treatment scores). For participants who presented with a PDSQ-T score of <50 (n=19), respective before ART, after ART, and 2-month mean follow-up scores on the PCL-C were 44.6, 26.6, and 27.1. For participants who presented with a PDSQ-T score of ≧50 (n=33), respective before ART, after ART, and 2-month mean follow-up scores on the PCL-C were 59.5, 33.9, and 31.0. Similar consistent and sustained treatment effects were observed for reductions in symptoms of depression across all subgroups examined (FIG. 3 p<0.001 for all comparisons with pre-treatment scores).

The instruments used in this study measured symptom burden, as opposed to deriving formal diagnoses. However, for the PCL-C, various cutoff scores exist for screening or diagnosis of PTSD among different settings. A score of ≧44 has been shown to maximize diagnostic efficiency. (Blanchard 1996). Using this cutoff score, respective percentages of participants meeting this diagnostic criterion of PTSD before ART, after ART, and at 2-month follow-up were 79% versus 17% versus 14% (FIG. 4, p<0.001 for all comparisons with pre-treatment scores).

Similar results were observed in subgroups classified by age, presenting PDSQ score, and number of reported traumas still impacting life. For the CES-D, scores of ≧16 are traditionally interpreted as suggestive of clinically significant depression. (McDowell, I., & Newell, C. (1996). Measuring Health, a Guide to Rating Scales and Questionnaires, 2nd ed. New York: Oxford University Press). Respective percentages of participants meeting this definition of depression before ART, after ART, and at 2-month follow-up were 90% versus 27% versus 33% (p<0.001 for all comparisons with pre-treatment scores).

For the PSQI, a score of >5 indicates clinically meaningfully disturbed or poor sleep. (Buysse 1989). Respective percentages of participants meeting this definition of sleep dysfunction before ART, after ART, and at 2-month follow-up were 69% versus 40% versus 42% (p<0.01 for all comparisons with pre-treatment scores). Therefore, while not formally diagnostic, treatment with ART resulted in substantial reduction in symptoms with a majority of participants scoring below diagnostic cutoff scores for PTSD, depression, and sleep dysfunction after treatment and at 2-month follow-up.

No serious adverse events judged to be related to treatment with ART were reported during the course of treatment or during 2-month follow-up. During the treatment period (1-5 sessions), minor unrelated adverse events reported included headache and dizziness (n=1), lightheadedness (n=1), tension headache (n=1), lack of motivation (n=1), and waking during the night (n=1). One participant reported mild depression after their first ART session which did not reoccur in subsequent sessions. A second participant reported feeling mildly depressed the day after an ART session.

As described above, 14 of 66 participants (21.2%) who completed ART did not have 2-month follow-up data, thereby representing a potential source of bias for the 2-month results. In ANCOVA adjusted for baseline PCL-C score, adjusted post-treatment means were similar between the 52 participants with follow-up data versus the 14 participants who completed treatment but did not have follow-up data (31.2 versus 28.7, p=0.43). Evidence of similar initial treatment response was observed for adjusted post-treatment mean scores on the CES-D (11.5 versus 12.4, p=0.76) and BSI (10.1 versus 10.5, p=0.86).

As illustrated above, substantial reductions in self-report symptoms of PTSD were observed (86% having scores below the diagnostic cut-point at 2 months), including depression, anxiety, and global physical and psychological symptoms, and improvements in trauma-related growth, sleep quality, and self-compassion after a median of 4 treatment sessions. Favorable results were consistently observed among all subgroups examined and at 2-month post-treatment follow-up. The majority of persons screened were judged to be eligible for the study, and approximately 83% of those enrolled were willing and able to complete the brief treatment regimen including completion of study questionnaires. In addition, no serious adverse effects were noted. Collectively, these results motivate future investigation of ART using controlled study designs, and possibly different clinical populations.

Within the realm of eye movement therapies, there is no consensus as to the therapeutic role of these movements. Existing hypotheses include those related to: (1) working memory (Andrade, J., Kavanagh, D., & Baddeley, A. (1997). Eye movements and visual imagery: A working memory approach to the treatment of post-traumatic stress disorder. British Journal of Clinical Psychology, 36, 209-233); (2) reciprocal inhibition (van den Hout, M., Muris, P., Salemink, E., & Kindt, M. (2001). Autobiographical memories become less vivid and emotional after eye movements. British Journal of Clinical Psychology, 40, 121-130); (3) detached processing (Lee, C. W., Taylor, G., & Drummond, P. D. (2006). The active ingredient in EMDR: Is it traditional exposure or dual focus of attention? Clinical Psychology and Psychotherapy, 13, 97-107); (4) inter-hemispheric integration (Christman, S. D., Garvey, K. J., Proper, R. E., & Phaneuf, K. A. (2003). Bilateral eye movements enhance the retrieval of episodic memories. Neuropsychology, 17, 221-229); (5) physiological effects (Sacka, M., Lempa, W., Steinmetz, A., Lamprecht, F., & Hofmann, A. (2008). Alterations in autonomic tone during trauma exposure using eye movement desensitization and reprocessing (EMDR)—Results of a preliminary investigation. Journal of Anxiety Disorders, 22, 1264-1271); (6) orienting response (MacCulloch, M. J., & Feldman, P. (1996). Eye movement desensitization treatment utilizes the positive visceral element of the investigatory reflex to inhibit the memories of post-traumatic stress disorder: A theoretical analysis. British Journal of Psychiatry, 169, 571-579); and (7) possible neurobiological mechanisms (Shapiro, F. (2001). Eye movement desensitization and reprocessing: Basic principles, protocols and procedures (2nd ed.). New York: Guilford; Stickgold, R. (2002). A putative neurobiological mechanism of action. Journal of Clinical Psychology, 58, 61-75).

The protocol-specified sets of eye movements have a calming effect that may help to disengage the sympathetic response (e.g. increased heart rate, chest tightness, sweating, etc.) associated with recall of the traumatic memory and facilitate a reciprocal inhibition allowing enhanced information processing. As the patient seeks (is directed) to re-envision the traumatic event and direct a new narrative, the patient is able to imagine replacing images and sensations associated with the traumatic memory with self-selected images and sensations that are more pleasing and palatable. During this stage, the lateral left-right eye movements may simultaneously activate both hemispheres to foster inter-hemispheric communication in a “problem-solving” manner similar to that used during rapid eye movement (REM) sleep.

While ART was delivered principally to resolve symptoms of PTSD, large concomitant reductions were observed for symptoms of depression and cognitive and somatic anxiety, along with marked improvements in trauma-related growth, sleep quality, and self-compassion. At least two possibilities exist for these widespread and somewhat “non-specific” effects. First, given that PTSD is associated with a range of psychological and physical comorbid conditions (Bisson 2007; Hoge 2007), “systemic” clinical benefits are to be expected so long as traumatic experiences of the patient are the principal underlying source of their psychopathology. On the other extreme, it is possible that, at least in part, “non-specific” effects were observed simply from subject interaction with therapists (e.g. the Hawthorne effect). The observation that the reported treatment effects were sustained at 2-month follow-up (which did not include interaction with a therapist) would argue against a broad Hawthorne effect.

Guidelines from the recent Institute of Medicine (IOM) report on effective treatments for PTSD unanimously recommend cognitive behavioral therapies and most guidelines recommend EMDR. (Institute of Medicine 2008; Seidler, G. H., & Wagner, F. E. (2006). Comparing the efficacy of EMDR and trauma-focused cognitive-behavioral therapy in the treatment of PTSD: a meta-analytic study. Psychological Medicine, 36, 1515-1522).

While there is considerable empirical evidence of treatment effectiveness of PTSD with CBT (e.g. Bryant, R. A., Moulds, M. L., Guthrie, R. M., Dang, S. T., & Nixon, R. D. V. (2003). Imaginal exposure alone and imaginal exposure with cognitive restructuring in treatment of posttraumatic stress disorder. Journal of Consulting and Clinical Psychology, 71, 706-712; Foa, E. B., Dancu, C. V., Hembree, E. A., Jaycox, L. H., Meadows, E. A., & Street, G. P. (1999). A comparison of exposure therapy, stress inoculation training, and their combination for reducing posttraumatic stress disorder in female assault victims. Journal of Consulting and Clinical Psychology, 67, 194-200; Marks, I., Lovell, K., Noshirvani, H., Livanou, M., & Thrasher, S. (1998). Treatment of posttraumatic stress disorder by exposure and/or cognitive restructuring. Archives of General Psychiatry, 55, 317-324; Resick, P. A., Nishith, P., Weaver, T. L., Astin, M. C., & Feuer, C. A. (2002). A comparison of cognitive-processing therapy with prolonged exposure and a waiting condition for the treatment of chronic posttraumatic stress disorder in female rape victims. Journal of Consulting and Clinical Psychology, 70, 867-879), and EMDR (e.g. Rothbaum, B. O., Astin, M. C., & Marsteller, F. (2005). Prolonged exposure versus eye movement desensitization and reprocessing (EMDR) for PTSD rape victims. Journal of Traumatic Stress, 18, 607-616; Taylor, S., Thordarson, D. S., Maxfield, L., Fedoroff, I. C., Lovell, K., & Ogrodniczuk, J. S. (2003). Comparative efficacy, speed, and adverse effects of three PTSD treatments: Exposure therapy, EMDR, and relaxation training Journal of Consulting and Clinical Psychology, 71, 330-338), these therapies (and other evidence-based modalities) may have significant limitations including: (i) lengthy treatment regimens (e.g. Schnurr, P. P., Friedman, M. J., Engel, C. C., et al. (2007). Cognitive behavioral therapy for posttraumatic stress disorder in women. A randomized controlled trial. JAMA, 297, 820-830) and (ii) significant post-treatment residual symptoms.

### Example 3

Example 3 illustrates the use of the device during ART therapy session rather than the therapists' hand or wand movements. It was unknown if the device would replicate the therapeutic results seen during traditional ART therapy sessions, and thus several sessions were conducted to evaluate the efficacy of the device. This example demonstrates that the device generates similar results to the hand movements of the therapist.

Patient #1 had a prior ART session using hand to track, and then a subsequent session using the device. The patient stated that the device worked the same as when the therapist had used her hand. The patient stated that their brain could not get back to the other images. The patient also stated that the device took the variation out of the movement and was very consistent.

Patient #2 had an ART session utilizing the device and commented on the calming effect.

Patient #3 had an ART session utilizing the device and commented that it had worked. It changed her feeling about the problem and it was no longer an issue.

Patient #4 had an ART session utilizing the device and commented that it was extremely helpful and would use it again. This patient scored close to a “10” on the intensity of her problem and went down to less than a “1” after processing using the device.

Patient #5 had an ART session utilizing the device (first time with ART Therapy) She was able to process her anxiety issue from a “7” on the intensity scale down to a “0” and was pleased with session results. She had no experience with prior ART.

Patient #6 had an ART session utilizing the device and commented on the consistency of the device movement. The patient found no difference in the therapy results when using the device versus the clinician's hand.

From this assessment, ART appears to be a brief, safe, and effective treatment for symptoms of PTSD and related psychological comorbidities. The use of the device described herein produces the same results as ART therapy in which the therapist's hand is used as the object to be tracked. The device described herein saves the therapist from strain on their arm. Additionally, the device described herein may be used by the subject to process out minor issues without the presence of a therapist.

### Example 4

In addition to the devices described above, computer software has been developed which may be used on any standard computer. This software provides instructions for a computer processor to generate a moving graphic indicia on a computer display. The instructions are to display the graphic indicia on the computer display; to move the graphic indicia on a horizontal plane in a first direction until a first predetermined vertical boundary is met at which time the direction the graphic indicia is reversed to move in the opposing direction until it meets a second predetermined vertical boundary wherein it again reverses direction. This back and forth movement is a cycle and the cycle is repeated a predetermined number of times.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. A device for stimulating eye movement in a subject comprising:
a housing having a first end and a second end;
a motion mechanism disposed within the housing; and
an object permanently or removably attached to the motion mechanism;
wherein activation of the motion mechanism moves the object horizontally back and forth between the first and second ends of the housing;
whereby a subject follows the horizontal movement of the object for a predetermined number of cycles to stimulate eye movement and process negative sensations.

2. The device of claim 1, wherein the motion mechanism is a pulley system.

3. The device of claim 2, further comprising:
a first pulley disposed within the first end of the housing;
a second pulley disposed within the second end of the housing wherein the second pulley is rotatably attached to the motor;
a circular belt extending between the first pulley and the second pulley; and
wherein activation of the motor rotates the second pulley to move the belt in one direction and wherein movement of the belt rotates the first pulley in the same direction as the rotation of the second pulley.

4. The device of claim 1, further comprising at least one stand positioned beneath the housing wherein the object is positioned at eye level with the subject.

5. The device of claim 1, further comprising a slot disposed along the first side of the housing wherein the slot extends from substantially the first end of the housing to substantially the second end of the housing.

6. The device of claim 1, further comprising a motor attached to the motion mechanism.

7. The device of claim 6, further comprising a controller to provide voltage to the motor.

8. The device of claim 7, further comprising a power source electrically connected to the controller.

9. The device of claim 7, further comprising a microprocessor electrically interfaced with the controller.

10. A device for stimulating eye movement in a subject comprising:
a control component having a top side and a bottom side;
a microprocessor contained within the control component;
a visual component positioned on the top side of the control component;
a power source electrically attached to the control component; and
a plurality of switches on the top side of the control component wherein the plurality of switches send a signal to the microprocessor to control selecting and modulating the appearance and speed of an object projected on the visual component and tracking movement of the object on the visual component;
wherein the object moves in a substantially horizontal line across the visual component;
whereby a subject follows the horizontal movement of the object for a predetermined number of cycles to stimulate eye movement and process negative sensations.

11. The device of claim 10, further comprising a plurality of speakers disposed within the control component.

12. The device of claim 11, further comprising a plurality of switches on the top side of the control component for controlling the volume and type of sound emanating from the speakers.

13. The device of claim 10, wherein the power source is a battery.

14. A computer-implemented therapeutic software application, the software accessible from a non-transitory media and providing instructions for a computer processor to generate a moving graphic indicia on a computer display, the instructions comprising:
displaying the graphic indicia on the computer display;
moving the graphic indicia on a horizontal plane in a first direction;
responsive to the graphic indicia meeting a first predetermined vertical boundary of the computer display, reversing the direction the graphic indicia to move in the opposing direction until it meets a second predetermined vertical boundary wherein it again reverses direction, the back and forth movement comprising a cycle; and
repeating the cycle a predetermined number of times wherein an end user observing the cycling graphic indicia receives therapeutic benefit.

15. A method of stimulating eye movement in a subject comprising:
providing a device with an object visually displayed at eye level of the subject wherein the device moves the object horizontally in a first direction to a first predetermined stop point wherein at the first stop point the device moves the object in a second opposite direction to a second predetermined stop point with this back and forth movement comprising a cycle;
instructing the subject to follow the object with only their eyes;
repeating the cycle for a predetermined number of times wherein the number of times is between about 20 and about 40; and
assessing resolution of a problem;
wherein if the problem is not resolved, the cycle is repeated.

16. The method of claim 15, wherein the object is moving at a predetermined speed.

17. The method of claim 15, wherein the object is a physical object.

18. The method of claim 15, wherein the object is a computer-generated object.
